(19) United States
(12) Patent Application Publication
(10) Pub. No.: US 2019/0302119 A1
(43) Pub. Date: Oct. 3, 2019

(54) Cancer Diagnostic Metastasis Panel

(71) Applicant: Lawrence Abraham, Miami, FL (US)

(72) Inventor: Lawrence Abraham, Miami, FL (US)

(21) Appl. No.: 15/936,749

(22) Filed: Mar. 27, 2018

(51) Int. Cl. G01N 33/574; G16H 50/20; G16H 50/30; G01N 33/53; G06F 17/18; G06N 7/00

(52) U.S. Cl. CPC G01N 33/5748; G16H 50/20; G06N 7/005; G01N 33/5308; G06F 17/18; G16H 50/30

(57) ABSTRACT

There are three current challenges to checking Cancer metastasis. These challenges mitigate against effective development and design of a comprehensive and appropriate utility diagnostic. The challenges include: a) Origin of Circulating tumor cells (CTC) and Tumor spread mechanism of dissemination), b) The role of normal tissue in metastasis, and c) Resistance to drug. This present invention is a comprehensive diagnostic that provides a method for predicting and diagnosing Cancer spread, as well as determine efficacy of therapy in a patient sample. The Cancer Diagnostic Metastasis Panel (CDMP) is a dialogic invention that is also a reflection of key communication variables of intercellular and intra-cellular agents, based on cell behavior including non-adherence or adherence to ‘Tradition’; as a shared behavioral practice performed repeatedly over time, that depends in part on socially mutually aided learning for its generation in and among cells and in specific environment (see below), incapacity of the Host Defense System as well as perceptions of the changing character of the environments. There is a system of incentives actuated by conditions of nutrient poverty and dependency. Morphologically, new structures contend with the old.

The Cancer Diagnostic Metastasis Panel (CDMP) relies on the spontaneous recognition of the kinetics of the Circulatory System, and the almost spontaneous selection response of the Host Defense System (Immunologic System) in specific microenvironment based on incapacity; especially proximal (near) to the parent tumor cell. There is an energy to energy correlation measurable by energy-time specific levels and wavelengths. The spontaneity is enabled by signal energy resonance in the two systems (apple to apple) in recognition of the changing character of a field or microenvironment.

There are key elements which monitor the dialogic environment, and measurable benchmarks which serve as biomarkers.

The invention (apparatus) is a panel of cell circulating cancer tumor test, as well as group and pathway tests. The net effect of a comprehensive test panel is a ‘hear and learn’ process (kinetics and signaling); separating cause and effect, with the notable fact that the cells can co-act with the microenvironment to produce different pathways; with emphasis on CTC traffic (nature and energy level(s)) as well as environment. The removal of one element from the menu has significance for therapy because the environment may thus have been altered, hence inducing a ‘mismatch’ and effect on timing of drug release. The import of co-acting variables cannot be over-emphasized. However, the expert may deliberate alter the ‘environment’ to determine impact and efficacy of therapy. The communication menu is interdisciplinary in approach, drawing from selective signaling and kinetics.

For predictive purposes; the Cancer Diagnostic Metastasis Panel (CDMP) relies on the data information on the kinetics of the Circulatory System, and the almost spontaneous selection response by the Host Defense System (Immunologic System) to this movement (circulatory system) in specific microenvironment; especially proximal (near) to the parent tumor cell. The selection process in the Host Defense System relies on communication between the weakest cell (connected to the waste system) and the ‘lead managing cell’ (LEM1) of the impending circulating tumor cell (CTC), particularly when an overwhelmed Host Defense System (HDS) needs relief. The communication between the weak cell and HDS signal receptor with LEM1 triggers the exit of the lead cell manager (LEM1). The LEM1 is functionally aided by a HDS signal receptor in the LEM1 role as ‘manager’ of the connection of cells that form the tumor extracellular matrix; which is the circulating tumor cells (CTC's). The CTC's are unorganized without LEM1. The strength of LEM1, as enabling metastasis lead is especially relevant to claim.

The model provides elements to help inform ‘prior’ toward a more enhanced ‘posterior’ distribution as in the Bayesian theorem. ‘Tradition’ is used broadly as behavioral practice that is routine and relatively enduring; performed repeatedly over time and shared among two or more members of a group. The action depends in part on socially mutually aided learning for its generation in new practitioners (cells). The cells may act individually or collectively with the microenvironment to create other pathway(s). This Cancer Diagnostic Metastasis Panel (CDMP) is a ‘Tradition’ based (circulating tumor cell) detection measure that also allows the expert to assign weights to elements for use in testing.

The CDMP can be used to interpret significance of levels of inter- and intra-cellular activities as well as variable(s) impact of and on microenvironment; which measure and interpretive significance is a measure of cell(s) innovative response to the character of a cancerous field or micro-environment evident in the panel calculated results. One and the same cancerous cell or group of cells can engage in completely different actions in different fields, and it is not a priori possible to determine what the resulting action will be without a comprehensive panel list to determine or at least define the character of the field or tumor microenvironment; including especially, but not limited to the kinetics of the new field of activity and associated energy-time specific Circulatory and Host Defense System response.

The apparatus comprises A) Measure of Environmental Toxicity: levels of trace elements, differences in tissue characteristics (abnormality, stickiness, shape, mass, appearance after repeated interaction with tumor signals), level of oxygen (hypoxia), cell nutrients available (angiogenesis), B) Measure of Strategic Panel field Management: relationship between normal tissue cell and tumor cells, level of intercellular communication between tumor cell and non-tumor cells, intra-cellular communication, relationship between original parent tumor cell and daughter cells (driving agents), strength of driving agent cells, relationship between parent tumor cell and other cancer cells, C) Measure of Signaling Activity: surround cell toxicity, tumor cell form (including stability), tumor cell behavior, materiality changes (protein content change, nature and level), changing genes, resonant repetitive behavior of cancer cells vs. non cancer cells, T Cells signaling levels, D) Panel has saved space for storing results for reference, F) Measure of Strength of LEM1 lead managing cell of the extra cellular matrix corresponding to origin of CTC, G) Measure of Strength of receptor EM1 and EM2 (see below) and H) Cell and Cell cluster energy.

The panel is a method for computing metastasis;—said method comprising gathering, analyzing, calculating, and storing of computational data including aforementioned variables to calculate probability of metastasis using Bayesian theorem. The panel reflects individual and collective effects of variables in tumor microenvironment, effects of variables on other variables (interrelatedness), tumor killer cell effect on system. In agreement with claim (1), the panel of variables and calculations reveal the effect of the removal/substitution (adjustment) of variables to establish prognostic and predictive pharmacodynamics biomarker(s); with corresponding impact on the efficacy of drug therapy.

The apparatus incorporates strength measure of ‘LEM1’ in relation to management of migration of CTC's to another host site—as a functional measure—which initial migration is re-directed by host receptor EM1 to signal receptor EM2 which further directs to a more amenable site for wider metastasis. The ‘LEM1’ relies significantly on receptor cells both in and outside the Host Defense System (HDS) for this migration to a new site, which migration is central to the function of the variables in the panel. The strengths of EM1 and EM2 are relevant to accuracy of prediction.

The apparatus in its comprehensive aspect is useful in measuring stages of metastasis due to changes in probability resulting from increased signal activities; in the form of derivative input from results of biopsies or other means. Rapid metastasis sites are regions of interconnected signal networks or meeting point; a sort of network of networks (super-network) where messages are readily stored, and also developed, relayed and easily shared in a permissive environment, weakly defended. This low immune environment or region with little resistance to cell to cell as well as cell cluster communication; and with carcinogenic presence, is the original nexus for distal (far) or wider metastasis, measurably relevant.

The panel by a measure of transport kinetics can establish observable differences between cell mating signaling and other form of signaling indicating the factor of speed, time and ease as significant elements of metastasis, especially in environments conducive to metastasis. The measure of ultimate aim is a measure of migration to the original parent tumor cell. This comprehensive invention (apparatus) can be used to measure effect of rate or slowness in the exit of a cell from transport system and the impact on rate of metastasis. Test Monitoring can be affected at different wavelengths. The CDMP employs the use of Bayesian theorem and is unitarily deliberate in its gauge of statistical variable interdependencies. This invention (CDMP) can comparatively interpret results of resonant light wave characteristics and energy in cancerous and non-cancerous cells by comparing measured chromosomatic light effects on the cell(s) nutrients and gauging associated changes in the panel's playing field.

Regarding group cluster and cohesion; the cancerous/non-cancerous tissue communication matter as much as the communication between the tissue non-cancerous/non-cancer communications in consonance with reliability of probability. There is an ‘offspring’ to ‘original’ relation in Metastasis.

The relations between cancerous and non-cancerous cells are not necessarily adversarial, but mistrustful. It is more a question of identity and de-individuation. The panel field takes note of how these relations function, as reflected in gauge of final probability. The vulnerable non-cancerous tissue cell needs to negotiate quantifiable nutrient supply with the tumor cell. The tumor cell has the disadvantage of not being able to see through tissue cell without magnification (light).

This is an issue of recognition. Cell instability results because of a search for systemic balance. In instances of metastasis, the non-cancerous tissue usually has low immunity, and is constantly appraising estimated need discernible by the co-action of the variables. The level of signaling activity between normal tissue cells and Circulating Tumor Cells (CTC's) are co-dependent in interaction. (This type of interaction also affects tumor dormancy). The strength level of signaling acts as biomarker, and is thus identifiable and measurable in substitutive element.

This comprehensive apparatus (Cancer Diagnostics Metastasis Panel) provides for measure of polarity snapshot of the medium or media of interactions. The efficacy of intermediate stage interaction with an acceptor or different acceptors, as enablers to traditional behavior modifications can be established with the CDMP. At the interstitial level of interaction, there is recognition and signal monitor of the original and the transitory (intermediate) stage involved in a ‘culture’ interaction that is also a factor in the multiplication of cells. The cancer cells are not adhering to non-metastasis tradition in dealing with non-cancerous cells. The kinetic consequence of an intermediate may be used to diagnose its value in metastasis, which effect (s) can be seen in the changes in panel measure.

This comprehensive apparatus in its panel form functions as a distinguishing dialogic/communicative enabler or reflector. For example, the INSR as a cell membrane receptor is the bridge of understanding able not only to transmit, but also mold messages to and from outside the cell;—a level of entrepreneurship measurable by deliberately changing nutrient content corresponding to level of mediating signaling activity, and measuring effect on overall probability of metastasis.

This comprehensive apparatus is useful in interpreting and recording the traffic characteristics of the circulatory system as a significant biomarker in metastasis. These characteristics include CTC traffic energy over time, speed, effectiveness of control devices over CTC's, and also flow. There is a flow vs. concentration (density) factor in metastasis; especially, but not limited to the kinetic and signal wave character of the circulatory system. Circulating tumor cells (CTC) cells spread and recruit into an alliance (pathway) depending on the environment or culture by undermining the immune system. The panel is useful in evaluating effect of this movement on metastasis.

Cancerous cells will often operate provincially within a pathway, and also in the context of a collective facilitation or recruitment of vulnerable cells into a cancer pathway through selective content change (nutrients, oxygen, and glucose). The panel is useful as a trend measure. This Comprehensive Apparatus and Method Diagnostic is also a measure of the impact of any element(s) changes on individual and group cell behavior; especially cell variables that would disrupt expected behavior relative to cohesion.

The number and nature of co-acting variables impact the timing of drug release, which effect is evident as a collective measure of significance of variables in the panel field.

1) This invention is a functional utility with associated variables. This invention is a comprehensive panel apparatus that is useful in monitoring and predicting spread of circulating tumor cells (CTC's) and changes in tumor microenvironment. This Diagnostic Apparatus (CDMP) is functionally geared toward high degree predictive probability of metastasis as well as monitoring the efficacy of drug therapy. For enhanced accuracy, the Diagnostic is enabled by dependent component variables, acting and co-acting as one panel; said comprehensive apparatus comprising rows and columns of panel field variables obtained through biopsies or other means. The mechanics of the variables' effect are important to panel accuracy. The CDMP can be used to interpret significance of levels of inter- and intra-cellular activities as well as variable(s) impact of and on microenvironment; which measure and interpretive significance is a measure of cell(s) innovative response to the character of a cancerous field or micro-environment evident in the panel calculated results. One and the same cancerous cell or group of cells can engage in completely different actions in different fields, and it is not a priori possible to determine what the resulting action will be without a comprehensive panel list to determine or at least define the character of the field or tumor microenvironment; including especially, but not limited to the kinetics of the new field of activity and associated energy-time specific Circulatory and Host Defense System response.

2) According to claim (1), the apparatus is comprised, but not limited to Rows: A) cell count (including T cells), rate of cell apoptosis, constitutive blood cells (platelets,—basophils, eosinhils, lymphocytes, process hematopoiesis), B) Measure of Environmental Toxicity: levels of trace elements, differences in tissue characteristics (abnormality, stickiness, shape, mass, appearance after repeated interaction with tumor signals), level of oxygen (hypoxia), cell nutrients available (angiogenesis), C) Measure of Strategic Panel field Management: relationship between normal tissue cell and tumor cells, level of intercellular communication between tumor cell and non-tumor cells, intra-cellular communication, relationship between original parent tumor cell and daughter cells (driving agents), strength of driving agent cells, relationship between parent tumor cell and other cancer cells, D) Measure of Signaling Activity: surround cell toxicity, tumor cell form (including stability), tumor cell behavior, materiality changes (protein content change, nature and level), changing genes, resonant repetitive behavior of cancer cells vs. non cancer cells, T Cells signaling levels, E) Panel has saved space for storing results for reference, F) Measure of Strength of LEM1 lead managing cell of the extra cellular matrix corresponding to origin of CTC, G) Measure of Strength of receptor EM1 and EM2 (see below) and H) Cell and Cell cluster energy tests.

3) According to claim (1), the panel is a method for computing metastasis;—said method comprising gathering, analyzing, calculating, and storing of computational data including aforementioned variables to calculate probability of metastasis using Bayesian theorem. The panel reflects individual and collective effects of variables in tumor microenvironment, effects of variables on other variables (interrelatedness), tumor killer cell effect on system. In agreement with claim (1), the panel of variables and calculations reveal the effect of the removal/substitution (adjustment) of variables to establish prognostic and predictive pharmacodynamics biomarker(s); with corresponding impact on the efficacy of drug therapy.

4) According to claim (1), for predictive purposes; the Cancer Diagnostic Metastasis Panel (CDMP) relies on the data information on the kinetics of the Circulatory System, and the almost spontaneous selection response by the Host Defense System (Immunologic System) to this movement (circulatory system) in specific microenvironment; especially proximal (near) to the parent tumor cell. The selection process in the Host Defense System relies on communication between the weakest cell (connected to the waste system) and the ‘lead managing cell’ (LEM1) of the impending circulating tumor cell (CTC), particularly when an overwhelmed Host Defense System (HDS) needs relief. The communication between the weak cell and HDS signal receptor with LEM1 triggers the exit of the lead cell manager (LEM1). The LEM1 is functionally aided by a HDS signal receptor in the LEM1 role as ‘manager’ of the connection of cells that form the tumor extracellular matrix; which is the circulating tumor cells (CTC's). The CTC's are unorganized without LEM1. The strength of LEM1, as enabling metastasis lead is especially relevant to claim (1).

5) According to claim (1), the apparatus incorporates strength measure of ‘LEM1’ in relation to management of migration of CTC's to another host site—as a functional measure-which initial migration is re-directed by host receptor EM1 to signal receptor EM2 which further directs to a more amenable site for wider metastasis. The ‘LEM1’ relies significantly on receptor cells both in and outside the Host Defense System (HDS) for this migration to a new site, which migration is central to the function of the variables in the panel. The strengths of EM1 and EM2 are relevant to accuracy of prediction.

6) According to claim (1), the apparatus in its comprehensive aspect is useful in measuring stages of metastasis due to changes in probability resulting from increased signal activities; in the form of derivative input from results of biopsies or other means. Rapid metastasis sites are regions of interconnected signal networks or meeting point; a sort of network of networks (super-network) where messages are readily stored, and also developed, relayed and easily shared in a permissive environment, weakly defended. This low immune environment or region with little resistance to cell to cell as well as cell cluster communication; and with carcinogenic presence, is the original nexus for distal (far) or wider metastasis, measurably relevant as in claim (1).

7) According to claim (1), the panel by a measure of transport kinetics can establish observable differences between cell mating signaling and other form of signaling indicating the factor of speed, time and ease as significant elements of metastasis, especially in environments conducive to metastasis. The measure of ultimate aim is a measure of migration to the original parent tumor cell. This comprehensive invention (apparatus) can be used to measure effect of rate or slowness in the exit of a cell from transport system and the impact on rate of metastasis. Test Monitoring can be affected at different wavelengths. The CDMP employs the use of Bayesian theorem and is unitarily deliberate in its gauge of statistical variable interdependencies. This invention (CDMP) can comparatively interpret results of resonant light wave characteristics and energy in cancerous and non-cancerous cells by comparing measured chromosomatic light effects on the cell(s) nutrients and gauging associated changes in the panel's playing field.

8) According to claim (1), the apparatus as a panel comprises the measure of means, mode and language of variables' communication as well as associated impact on speed (velocity) of the social cell learning process in addition to the mass of tumor cells. Regarding group cluster and cohesion; the cancerous/non-cancerous tissue communication matter as much as the communication between the tissue non-cancerous/non-cancer communications in consonance with reliability of probability, as in claim (1). There is an ‘offspring’ to ‘original’ relation in Metastasis.

9) According to claim (1), the apparatus' measure of cell stability depends on relations between cancerous and non-cancerous tissue cells. The relations are not necessarily adversarial, but mistrustful. It is more a question of identity and de-individuation. The panel field takes note of how these relations function, as reflected in gauge of final probability. The vulnerable non-cancerous tissue cell needs to negotiate quantifiable nutrient supply with the tumor cell. The tumor cell has the disadvantage of not being able to see through tissue cell without magnification (light). This is an issue of recognition. Cell instability results because of a search for systemic balance. In instances of metastasis, the non-cancerous tissue usually has low immunity, and is constantly appraising estimated need discernible by the co-action of the variables. The level of signaling activity between normal tissue cells and Circulating Tumor Cells (CTC's) are co-dependent in interaction. (This type of interaction also affects tumor dormancy). The strength level of signaling acts as biomarker, and is thus identifiable and measurable in substitutive element as in claim (1).

10) According to claim (1), the comprehensive apparatus (Cancer Diagnostics Metastasis Panel) provides for measure of polarity snapshot of the medium or media of interactions. The efficacy of intermediate stage interaction with an acceptor or different acceptors, as enablers to traditional behavior modifications can be established with the CDMP. At the interstitial level of interaction, there is recognition and signal monitor of the original and the transitory (intermediate) stage involved in a ‘culture’ interaction that is also a factor in the multiplication of cells. The cancer cells are not adhering to non-metastasis tradition in dealing with non-cancerous cells. The kinetic consequence of an intermediate may be used to diagnose its value in metastasis, which effect (s) can be seen in the changes in panel measure.

11) According to claim (1), the apparatus does factor in the presence of mediating signals (such as INSR; IRS-1) as proprietary and self-interested in that they facilitate or hinder communication, as well as reinterpret and direct the message especially in weak or diseased cells;—relative to measure of level of signaling activity. Essentially, the cells hear and learn through bargaining and mediation. In metastasis, circulatory energy and wavelength matter. Regarding Cancer Tumor Cell (CTC's), weak cells in train or transport slow metastasis.

12) According to claim (1); the apparatus, in its method takes cognizance of the cell energy test in relation to cellular respiration and hypoxia and its (cell energy) utilization in the form of adenosine triphosphate (ATP) molecules; as well as the metabolic network. In this regard, the T-cell energy levels are significant biomarkers.

13) According to claim (1), this comprehensive apparatus in its panel form functions as a distinguishing dialogic/communicative enabler or reflector. For example, the INSR as a cell membrane receptor is the bridge of understanding able not only to transmit, but also mold messages to and from outside the cell;—a level of entrepreneurship measurable by deliberately changing nutrient content corresponding to level of mediating signaling activity, and measuring effect on overall probability of metastasis.

14) According to claim (1), this comprehensive apparatus can indicate corresponding impact of changes on overall metastasis observable in cell metabolic content changes, cell stability changes (form), changes in cell behavior, as biomarkers; with increased level of field or microenvironment kinetic and signaling activities.

15) According to claim (1), this apparatus and method diagnostic evaluates level of circulating tumor cell behavior within a culture (environment) as well as recruitment into a cancer alliance pathway. The invention can be used functionally to monitor cell culpability and exchange by a radical alteration of nutrient content so as to determine expected and unexpected behavior.

16) According to claim (1), this comprehensive apparatus can interpret and record the traffic characteristics of the circulatory system as a significant biomarker in metastasis. These characteristics include CTC traffic energy over time, speed, effectiveness of control devices over CTC's, and also flow. There is a flow vs concentration (density) factor in metastasis; especially, but not limited to the kinetic and signal wave character of the circulatory system. Circulating tumor cells (CTC) cells spread and recruit into an alliance (pathway) depending on the environment or culture by undermining the immune system. The panel is useful in evaluating effect of this movement on metastasis.

17) According to claim (1), this apparatus and method can measure effects on spread of non-tumor tissue cells manipulation by cancerous cells through communication via regulatory agency of the microenvironment (level of sodium, potassium, calcium).

18) According to claim (1), this diagnostic apparatus measures pathway inter-cancer cells group security in relation to parent tumor cell; and the nature of inter-cellular communication is important to metastasis. Cancerous cells will often operate provincially within a pathway, and also in the context of a collective facilitation or recruitment of vulnerable cells into a cancer pathway through selective content change (nutrients, oxygen, and glucose). The panel is useful as a trend measure.

19) According to claim (1), this Comprehensive Apparatus and Method Diagnostic is also a measure of the impact of any element(s) changes on individual and group cell behavior; especially cell variables that would disrupt expected behavior relative to cohesion.

20) According to claim (1), this Diagnostic Apparatus facilitates monitoring of effects of drug therapy. A change in any of the variables in the panel field might alter the playing field. This is significant with regards to drug therapy and avoidance of mismatch, and targeting of running targets. The number and nature of co-acting variables impact the timing of drug release, which effect is evident as a collective measure of significance of variables in the panel field.
